(19) United States
(12) Patent Application Publication
(10) Pub. No.: US 2006/0263511 A1
(43) Pub. Date: Nov. 23, 2006

(54) Lens insertion system

(76) Inventors: Steven Musch, Rapid City, SD (US); Douglas Mastel, Rapid City, SD (US); Mark C. Gross, Chaska, MN (US); Daniel McNamara, Richfield, MN (US)

Correspondence Address: Gene R. Woodle, 3516 Woodle Drive, Rapid City, SD, 57702, US

(21) Appl. No.: 11/435,177

(22) Filed: May 15, 2006

Related U.S. Application Data
(60) Provisional application No. 60/672,132, filed on May 19, 2005.

(51) Int. Cl. A61L 33/00

(52) U.S. Cl. 427/2.1

(57) ABSTRACT

A intra-ocular lens insertion system and a method for coating a cartridge for use with such a system is disclosed. The coating method dicloses means for applying a two layer coating having a base coat and a top coat to the interior of a lens insertion cartridge which promotes lubricity.

### RELATED APPLICATIONS

This application relies for priority upon the Provisional Patent Application filed by Steven Musch, Douglas Mastel, and Mark C. Gross entitled Lens Insertion System, Ser. No. 60/672,132, filed May 19, 2005.

### BACKGROUND OF THE INVENTION

1. Field of the Invention

This invention relates generally to artificial lenses for placement in eyes, and more particularly to a system for insertion of such lenses into the eye of a patient.

2. Background Information

In this country and throughout the world, surgeons replace the lenses in the eyes of thousands of patients with artificial lenses. There are a wide variety of lenses and surgical techniques, but most often an artificial lens having a diameter of about 6 mm is placed in the eye through an incision which is approximately half that length. Because the lens is wider than the incision, the lens must be folded in half before it is inserted into the eye. The lens is usually inserted into and affixed within an open area of the eye known as the capsular bag which has a diameter of approximately 11.75 mm. In order to center the artificial lens in this capsular bag, artificial lenses often have hair-like “springs” known as haptics affixed to the perimeter of the lens such that they protrude outward from the lens. In some cases the haptic takes the form of a plurality of plates which protrude from the lens rather than the form of the fine spring haptic.

For a variety of reasons including the delicacy of an artificial lens, the delicacy of haptics, and the need to protect the eye of the patient; the insertion of the folded lens through the incision and into the eye has created a significant number of problems during the course of lens replacement surgery. The lens itself may be marred, blemished, or wrinkled, the haptics may be damaged, or the eye of the patient could actually be damaged. Through no fault of the surgeon, some of these potential defects are not readily apparent at the time of insertion and could lead to further damage to the eye over time. Some types of artificial lenses such as those containing a large percentage of silicone tend to unfold with such rapidity after insertion into the eye that the force of unfolding can cause injury to the lens or to the eye.

There are a number of lens insertion devices currently in use. A popular insertion device includes a plastic cartridge with a cylindrical tube at one end and a pair of wings at the other. With the wings open, a lens is placed in a cavity having a semicircular cross section at the base of the wings with the concave side of the lens up. The wings are then folded together which folds the lens into the shape of a taco and encloses the lens into a hollow tube. The cartridge is then placed in a handpiece which includes some type of plunger. The surgeon then inserts the tube through the incision into the eye and pushes the plunger. The end of the plunger pushed the lens through the tube and into the eye.

Experience has shown that there are a number of problems associated with such prior art devices. Although a number of lubrication methods have been tried, the friction associated with pushing the lens through the tube often causes damage to the lens. This friction also limits the ability of the surgeon to control the rate of insertion of the lens and the placement of the insertion. For instance, because of the force necessary to overcome the friction, the lens often pops out of the end of the tube in an uncontrolled manner. (This can be particularly problematic with silicone based lenses as pointed out above.) In addition to possible damage to the lens, such insertion devices provide no protection for delicate haptics and they are often damaged on insertion.

A number of inventions have attempted to address problems relating to intra-ocular lens insertion, applying a lubricious coating to plastic or, more specifically, to providing sufficient lubrication to a lens insertion cartridge for the lens to pass easily and smoothly through the hollow tube portion or lumen of the cartridge into the eye. The patent to Bartell (U.S. Pat. No. 4,681,102; Jul. 21, 1987) discloses a load chamber into which a lens may be placed and an injector which has a plunger to push the lens from the load chamber, through the tip of the injector and into the eye. The patent to Halpern et al. (U.S. Pat. No. 5,037,677; Aug. 6, 1991) discloses a method of applying an interlaminar coating onto glass or plastic. The patent claims applying a first coating to an object which contains an acrylic polymer and a solvent, removing the solvent, and adding a second coating which is an aqueous solution of sodium hyaluronate. The patent to Yang et al. (U.S. Pat. No. 5,803,925; Sep. 8, 1998) discloses a cartridge for the insertion of a lens in which the lubricity of the interior of the lumen is enhanced through covalently bonding a lubricity enhancing component to the interior wall of the lumen.

The instant invention is a lens insertion system which is unique, original, and solves all of the above noted problems relating to insertion of an intra ocular lens through an incision and into the eye.

The ideal lens insertion system should provide a method for inserting an artificial lens into the eye easily and efficiently without damage to the lens, the haptics on the lens, or to the eye. The ideal lens insertion system should allow the surgeon to insert, position, and align the lens easily and precisely. The ideal lens insertion system should allow for smooth and precise movement of the lens from the insertion device into the eye. The ideal lens insertion system should also be rugged, inexpensive, and easy to use.

### SUMMARY OF THE INVENTION

The lens insertion system of the instant invention comprises a handpiece and a cartridge. The handpiece has two major components: the plunger and the receptor.

The cartridge is made from a polymer and includes a hollow tube with a beveled tip at the forward end. The hollow tube is slit at the rearward end and there is a pair of wings which protrude upward from either side of this slit. Although the wings are vertical in normal aspect, they may be pulled apart to open the slit to create an opening in the hollow tube which is referred to as the loading zone. A lens is placed within the loading zone (usually concave side up, but there may be lenses which require different orientations as suggested by the manufacturer). If the lens includes two threadlike haptics as described above, one of the haptics is positioned such that it runs from the bottom edge of the lens forward into the hollow tube. The other haptic runs from the top of the lens out the back of the loading zone. As discussed above, the chances of inserting a lens into the eye without damage to the lens, the haptics, or the eye are greatly enhanced if the friction between the lens and the cartridge is minimized.

One of the major elements of the lens insertion system of the instant invention is a unique and original process for permanently coating the interior of the cartridge with a lubricant to greatly reduce the friction between the lens and the cartridge. Because the lubricious top coat can not be effectively affixed directly to the plastic interior of the cartridge, it is first necessary to affix a base coat to the desired areas of the interior of the cartridge. After the base coat has been applied and appropriately treated, the top coat is applied to the base coat and is permanently affixed to the base coat. The top coat is a solution of a polymer selected from the group consisting of a polysaccharide, a cellulose derivative, polyacrylic acid, and polyethylene glycol. The preferred top coat is an aqueous solution of hyaluronan. The top coat may also include surfactants, crosslinking agents, plasticizers, solvents, salts, or leveling agents. The base coat may be selected from any of the group of polymers or copolymers capable of adhering to the surface of the interior of the cartridge and to which the top coat may be affixed. Preferably the base coat is an acrylic polymer in an organic solvent.

Prior to placement of the lens in the loading zone, the interior of the cartridge should be lubricated with either a balanced salt solution (BSS) or viscoelastic (sodium hyaluronate). After the lens has been placed in the loading zone, the wings are folded back to their vertical position which causes the lens to fold into the shape of a taco and be enclosed within the rearward portion of the hollow tube. The cartridge is then inserted through a slot in the receptor such that the beveled end of the hollow tube protrudes through an opening in the forward end of the receptor and the cartridge is held in place within the receptor with the wings together. The plunger and the receptor are mated with threads at their rearward ends such that when a knob on the plunger is turned, the plunger moves forward within the receptor. The plunger has a specially configured tip at its forward end which comes into contact with the rear of the lens as the plunger is screwed into the receptor.

The beveled end of the cartridge is inserted through an incision in the eye and the handpiece manipulated by the surgeon until the beveled end of the cartridge is in the appropriate position within the eye. The surgeon then continues to turn the knob on the plunger which moves the plunger forward within the receptor. The tip of the plunger engages the rear of the lens and smoothly pushes the lens through the hollow tube and out into the appropriate location within the eye. The lens and any haptics unfold and the cartridge is removed from the eye.

One of the major objects of the present invention is to provide a lens insertion system for inserting an artificial lens into the eye easily and efficiently without damage to the lens, the haptics on the lens, or to the eye.

Another object of the present invention is to provide a lens insertion system which allows the surgeon to insert, position, and align the lens easily and precisely.

Another object of the present invention is to provide a lens insertion system which allows for smooth and precise movement of the lens from the insertion device into the eye Another object of the present invention is to provide a lens insertion system which is rugged, inexpensive, and easy to use.

These and other features of the invention will become apparent when taken in consideration with the following detailed description and the drawings.

### BRIEF DESCRIPTION OF THE DRAWINGS

FIG. 1 is a side view of the plunger of the lens insertion system of the instant invention;

FIG. 2 is a side view of the receptor of the instant invention;

FIG. 3 is a top view of a portion of the receptor of the instant invention;

FIG. 4 is a detail side view of the tip portion of the plunger of the instant invention;

FIG. 5 is an end view of the tip portion of the plunger of the instant invention;

FIG. 6 is a side view of the cartridge of the instant invention;

FIG. 7 is an end view of the cartridge of the instant invention;

FIG. 8 is a side view of the cartridge of the instant invention showing the loading of a lens into the cartridge;

FIG. 9 is a side view of the cartridge of the instant invention showing the lens being pushed through the cartridge with the tip portion of the plunger;

FIG. 10 is an end view of the cartridge of the instant invention in the plasma fixture prepared for plasma treatment;

FIG. 11 is a sectional view of the cartridge and plasma fixture taken along line 11-11 of FIG. 10;

FIG. 12 is a top view of the cartridge holder with the cartridge of the instant invention inserted into the cartridge holder; and

FIG. 13 is a sectional view of the cartridge holder and cartridge of the instant invention taken along line 13-13 of FIG. 12.

### DESCRIPTION OF A PREFERRED EMBODIMENT

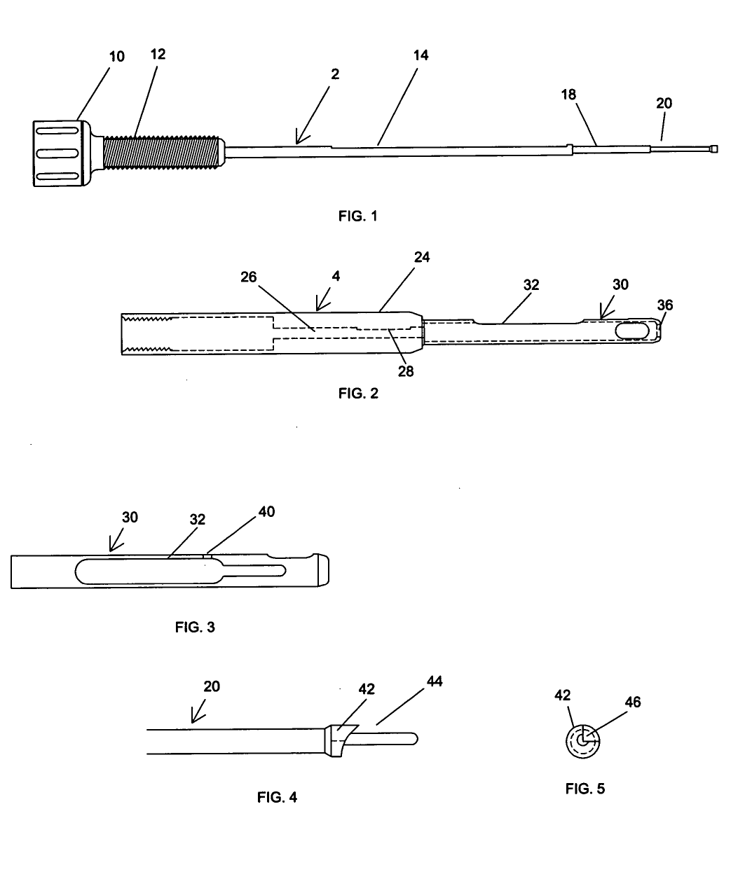

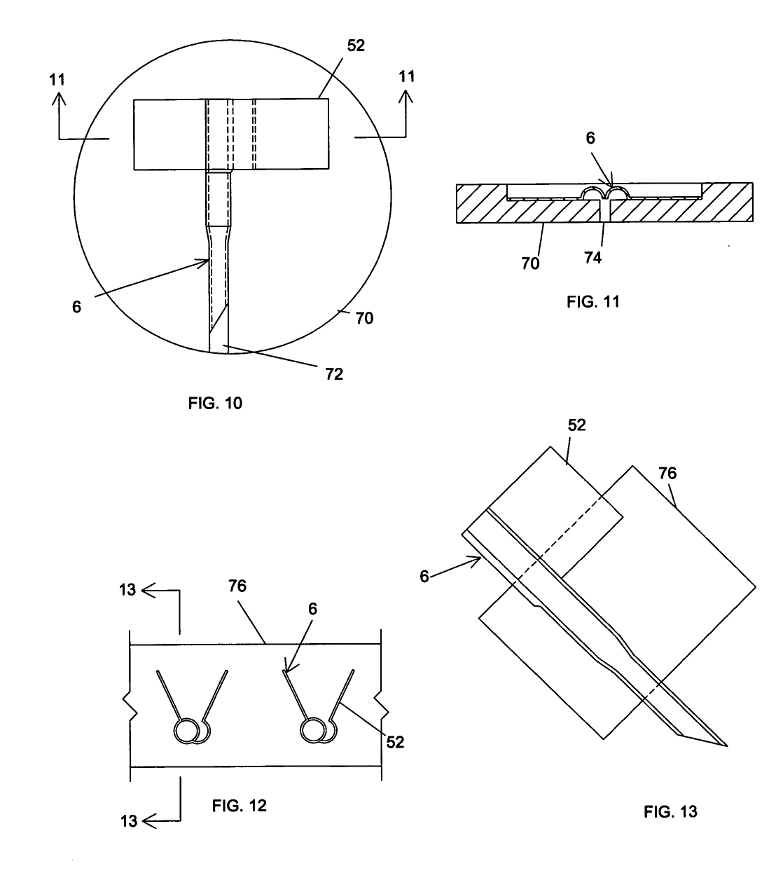

Referring to the drawings, FIGS. 1 through 9 shown a preferred embodiment of the lens insertion system of the instant invention. In addition some secondary embodiments are also shown and referenced. The instant invention includes three major elements: a plunger 2, a receptor 4, and a cartridge 6 which are referenced in various drawings. In general, the plunger 2 is permanently affixed chiefly within the hollow interior of the receptor 4 and the cartridge 6 may be removably affixed chiefly within the hollow interior of the forward end of said receptor 4. All of the elements of these major elements and their interaction are described below. FIGS. 10 through 13 show various elements used in the method of creating the bilaminar (two coat) coating applied to the interior of the cartridge of the instant invention.

Referring to the drawing FIG. 1, a side view of the plunger of the instant invention is shown. Said plunger 2 has a knob 10 at its rearward end. Just forward of the knob 10 is a shank 12 which is threaded. A shaft 14 protrudes forward from the shank 12. The shaft 14 is rotatably affixed to said shank 12. All of the elements of said plunger 2 are generally cylindrical, but the forward portion of said shaft 14 includes a flat 16 which runs nearly to the forward end of said shaft 14. A secondary shaft 18 protrudes forward from the forward end of said shaft 14 and a tip 20 protrudes forward from the forward end of the secondary shaft 18. The diameter of these elements generally decreases from rearward end to forward end.

Referring now to FIG. 2, a side view of the receptor of the instant invention is shown. Said receptor 4 is generally hollow and cylindrical in shape and has an collar 24 at its rearward end. The collar 24 is threaded at the interior of its rearward end and the threads mate with the threads of said shank 12 of said plunger 2. In the forward end of said collar 24 is a tube 26 which has an interior diameter slightly greater than the exterior diameter of said shank 12. Near the forward end of the tube 26 is a tube flat 28 which is complementary to the flat 16 on said shaft 14. Said plunger 2 and said receptor 4 are manufactured such that said shaft 14 is within said tube 26 and the ends of said flat 16 bracket the tube flat 28. Thus said shaft 14 may move freely forward and rearward within said tube 26, but the extent of the forward and rearward movement is restricted because the ends of said flat 16 engage the ends of said tube flat 28 and further forward or rearward movement is restricted by that engagement. Furthermore, because the inner surface of said flat 16 engages the inner surface of said tube flat 28, said shaft 14 may not rotate within said tube 26. A receptor tip 30 protrudes forward from the forward end of said collar 24. The receptor tip 30 is generally a hollow cylinder with a slot 32 opening into the interior of said receptor tip 30 near the middle of said receptor tip 30. There is a hole 36 at the forward end of said receptor tip 30. As may be seen, said plunger 2 fits within said receptor 4 and moves forward and rearward within said receptor 4. The threads on said shank 12 may be engaged with the threads of said collar 24 and said plunger 2 moved forward or rearward by turning said knob 10. Thus by manipulating said knob 10 the tip 20 may be moved such that its forward end may be moved from a position within the slot 32 to a position where its forward end protrudes forward from the hole 36.

Referring now to FIG. 3, a top view of a portion of the receptor of the instant invention is shown. This view shows the top of said receptor tip 36. Said slot 32 has a rearward portion which is significantly wider than its forward portion. A haptic slot 40 is cut into said receptor tip 36 which opens into said slot 32 just rearward of the narrow portion of said slot 32.

Referring now to FIG. 4, a detail side view of the tip portion of the plunger of the instant invention is shown. Said tip 20 has a pusher 42 at its forward end. The forward end of the pusher 42 is concave such that it conforms to the shape of the edge of a circular lens folded in half. A pusher tip 44 protrudes forward from the forward end of said pusher 42. This pusher tip 44 element may not be present in some embodiments of the instant invention.

Referring now to FIG. 5, an end view of the tip portion of the plunger of the instant invention is shown. This view shows the end of said pusher 42 and also shows that approximately one fourth of said pusher 42 is cut away to form a pusher haptic slot 46. This pusher haptic slot 46 puts the area forward of said pusher 42 in communication with the area rearward of said pusher 42 and, as will be shown in greater detail below, a haptic on a lens may be threaded through said pusher haptic slot 46.

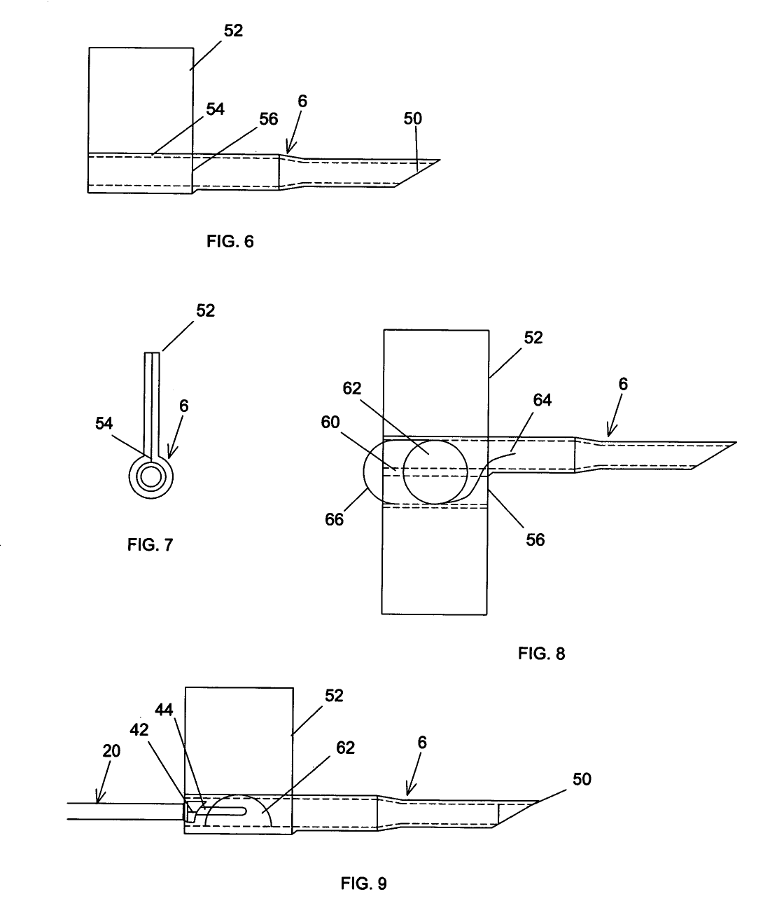

Referring now to FIG. 6, a side view of the cartridge of the instant invention is shown. The cartridge 6 has the general configuration of a hollow tube with a beveled end 50 at its forward end. The bevel of the beveled end 50 slants downward and rearward from the forward end of said cartridge 6. This is important as it insures that when a lens is pushed from said cartridge 6, it is in the proper alignment and position within the eye. A pair of wings 52 protrude upward from a slit 54 in the rearward end of the hollow tube of said cartridge 6. (This aspect of said cartridge 6 may be better seen in FIG. 7 discussed below.) There is a secondary slit 56 through the side wall of said cartridge 6 below the forward edge of said wing 52. In the preferred embodiment said cartridge 6 is made from polypropylene, but other materials having similar properties could be used.

Referring now to FIG. 7, an end view of the cartridge of the instant invention is shown. This view shows said wings 52 where they are affixed one either side of the slit 54.

Referring now to FIG. 8, a side view of the cartridge of the instant invention showing the loading of a lens into the cartridge is shown. This view shows how said wings 52 may be pulled apart to open up the rearward portion of said cartridge 6 along the secondary slit 56 to create what is referred to as a loading zone 60. A lens 62 with a leading haptic 64 and a following haptic 66 is placed concave surface up within the loading zone 60. The leading haptic 64 is threaded forward into the forward portion of the hollow tube of said cartridge 6. Said wings 52 may then be folded together (not shown) which captured the lens 62 within the loading zone 60 and folds said lens 62 into the shape of a taco.

Referring now to FIG. 9, a side view of the cartridge of the instant invention showing the lens being pushed through the cartridge with the tip portion of the plunger is shown. After said lens 62 has been captured within said cartridge 6 as described above, said cartridge 6 is inserted into said slot 32 (shown in FIG. 3) with said beveled end 50 forward and said cartridge 6 pushed forward such that said wings 52 are captured in the narrow portion of said slot 32 and said beveled end 50 protrudes forward through said hole 36 in said receptor tip 36. The following haptic 66 may be threaded rearward through the pusher haptic slot 46 and said haptic slot 40. Said knob 10 is turned until the concave face of said pusher 42 engages the rearward edge of said lens 62. Said beveled end 50 is inserted through an incision and into the eye at the appropriate location. Said knob 10 is then turned further which moves said pusher 42 forward pushing said lens 62 through said cartridge 6. Said pusher tip 44 engages the interior of said lens 62 which helps said lens 62 to maintain its shape to prevent tearing or distortion of said lens 62 as it is pushed through said cartridge 6. Said pusher tip 44 also grips said lens 62 by pressing said lens 62 between said pusher tip 44 and the interior wall of said cartridge 6. This gripping promotes control of the movement of said lens 62 through said cartridge 6 and prevents said lens 62 from popping out of the beveled end 50 in an uncontrolled manner. After said lens 62 has been pushed from said cartridge 6 and properly position within the eye, the lens insertion system may be removed from the eye.

As mentioned previously, a key element to the success of any lens insertion system is to minimize the friction between the lens and the interior of the cartridge. The lens insertion system of the instant invention includes a unique and original method of permanently coating the interior surface of the polymer cartridge with a lubricating material. The coating process involves applying two coatings, a base coat and a top coat, to the appropriate areas of the interior of the cartridge. The base coat is affixed to the cartridge first. The lubricating coating or the top coat is of some appropriate lubricating material which may be permanently affixed to the base coat. The top coat is a solution of a polymer selected from the group consisting of a polysaccharide, a cellulose derivative, polyacrylic acid, and polyethylene glycol. The preferred to coat is an aqueous solution of hyaluronan. The top coat may also include surfactants, crosslinking agents, plasticizers, solvents, salts, or leveling agents. The base coat may be selected from any of the group of polymers or copolymers capable of adhering to the surface of the interior of the cartridge and to which the top coat may be affixed. Preferably the base coat is an acrylic polymer. In the preferred embodiment of the instant invention this base coat and top coat is a material system known as Hydak (trademark) manufactured by Biocoat, Inc. Because the top coat cannot be made to effectively stick to the polymer from which the cartridge is made, a base coat must first be applied to the cartridge. The base coat sticks or bonds to the polymer and the top coat sticks or bonds to the base coat. In the preferred embodiment the base coat is an acrylic polymer, and the top coat is hyaluronic acid or sodium hyaluronate (and related Biocoat, Inc. coating derivatives). The coating process comprises the following steps:
1. The cartridge is visually inspected for defects and then cleaned with soap and with a solvent and dried.
2. The cartridge is inserted into a plasma fixture which holds the cartridge in the proper position and orientation such that an atmospheric plasma stream passes through the loading zone and then some plasma travels over the insides of the wings and the remainder through the hollow tube (or lumen). The plasma stream passes through a plasma fixture which insures that the plasma contacts only the portions of the cartridge which are intended to be coated with the base and top coat. The plasma stream conditions the polymer to promote sticking of the base coat, and masking to the cartridge.
3. Masking is applied by an applicator to the areas of the cartridge where neither base coat nor top coat is desired. In another embodiment solid masking may be applied with adhesive. That is, neither the base coat nor the top coat will stick to areas where the masking has been applied. The masking is applied to the inner surfaces of both wings, but not to the inner surfaces closest to the hollow tube of the cartridge. In the preferred embodiment of the instant invention, the 2 mm of the inner surface of the wings closest to the hollow tube are not coated with masking. The portion of the cartridge where the inner surface of the wing meets the hollow tube is referred to as the lip. The masking is allowed to cure.
4. The cartridge is inserted into a cartridge holder which holds the wings apart, allows manipulation of the cartridge, and leaves the interior of the cartridge exposed. The cartridge holder is inserted into a flipper which holds the cartridge holder and moves the cartridge holder and the cartridge to the various positions and orientations described below. Base coat is applied to the lip, the inner surface of the wings not coated by masking solution, and the loading zone. There may be instances where it may be appropriate to create or coat a cartridge which doesn't have lips. In such cases neither coat would, of course, be applied to any lips. The cartridge is held at about 45 degrees (this angle may vary from 30 to 60 degrees, depending upon the exact design of the cartridge being coated) while base coat is being applied with the beveled end of the hollow tube generally down. The cartridge is held in this position for approximately five seconds. (This time may vary depending upon cartridge configuration or materials used.) The cartridge is then tipped such that it is vertical with the beveled tip pointing down. The cartridge is held in this position for sufficient time to allow the base coat to run down the interior of the hollow tube. At the time when the base coat reaches the beveled tip of the cartridge, the cartridge is turned 180 degrees such that the beveled tip is pointing upward. Vacuum is applied to the loading zone end of the cartridge to remove excess base coat material from that area of the interior of the cartridge. Air is then blown through the beveled tip at sufficient pressure and for sufficient time to smooth the coating throughout the inner surface of the hollow tube, the loading zone, and the inner surface of the wings.
5. The cartridge in the cartridge holder is placed in an air distribution (preferably vacuum, however compressed air may be used) blocks inside a convection oven. The base coat is cured in a convection oven with vacuum drawing air through the hollow tube (lumen) of the cartridge. The amount of radiant heat applied in this step may be varied by regulating the fresh air intake of the convection oven and the heat radiating from the air distribution blocks holding the cartridge holder. In the preferred embodiment the curing time is twenty minutes at 60 degrees C., or as otherwise as defined by the coating manufacturer. In some cases (depending upon the base coat used or cartridge configuration) it may be preferable to use the combination of radiant heat drying and convection heat curing described in set 7 below.
6. The top coat is applied by placing the cartridge (still in its cartridge holder), back into the flipper and again tipping the cartridge to approximately 45 degrees (this angle may vary from 30 to 60 degrees, depending upon the exact design of the cartridge being coated). Top coat material is applied to the lip, the inner surface of the wings not coated by masking solution, and the loading zone. The cartridge is held in this position for approximately ten seconds (this time may vary depending upon material used and cartridge configuration) to allow the top coat to flow toward the hollow tube. This process takes somewhat longer because the top coat is more viscous than the base coat. The cartridge is tipped such that it is vertical with the beveled tip pointing down. The cartridge is held in this position for sufficient time to allow the top coat to run down the interior of the hollow tube. At the time when the top coat reaches the beveled tip of the cartridge, the cartridge is turned 180 degrees such that the beveled tip is pointing upward. After the coating has flowed down the lumen, vacuum is applied to the loading zone end of the cartridge to remove excess top coat material from that area of the interior of the cartridge. Air is then blown through the beveled tip at sufficient pressure and for sufficient time to smooth the coating throughout the inner surface of the hollow tube, the loading zone and the inner surface of the wings.
7. The top coat is then dried and cured in an oven. It is important to note that the top coat is dried using low humidity air and low heat. This method of drying prevents a “skin” from forming on the surface of the top coat. Formation of such a skin would prevent uniform evaporation of liquids from the top coat and would cause peeling, nonuniform coverage, and other defects in the top coating of the cartridge. In the preferred embodiment from 10% to 30% humidity and a temperature of about 30 degrees C. are maintained for about twenty minutes. When the top coat is dry, heat with blowing air is used to complete curing of the top coat for about two hours.
8. The cartridge is cooled and soaked in a bicarbonate solution to neutralize the coatings.
9. The cartridge is vigorously scrubbed to remove masking solution, excess base coat material, and excess top coat material. The cartridge is further treated with a conventional ultra sound cleaner to insure removal of all particulates.
10. The cartridge is rinsed to remove any particulates.
11. The cartridge is dried.

Although the above described coating process is described as being used for the lens insertion system cartridge of the instant invention, it could be used for other types, shapes, and sizes of objects with minimal modification or adjustments where a bilaminar or two layer coating needs to be placed on the interior of the object.

In a second embodiment, step 5 above may be altered somewhat. Most of the solvent in the base coat may be removed by inducing a flow of warm air through the interior of the cartridge ether by providing a vacuum or compressed air. The base coat is then cured in an oven as described above.

In a third embodiment, step 7 above may also be altered somewhat. Most of the solvent n the top coat may be removed by inducing a flow of warm air through the interior of the cartridge either by providing a vacuum or compressed air. The top coat is then cured in an oven as described above.

In a fourth embodiment, step 4 and step 6 may be altered somewhat to change the method by which the interior of the cartridge is coated with base coat and top coat. The cartridge is held in a vertical or near vertical position. Base coat or top coat is introduced into the interior of the cartridge either with or against gravity (from the top or from the bottom). Excess coating material is then removed from the interior of the cartridge either by allowing the coating to drain from the cartridge at a controlled rate or by inducing a flow or air through the interior of the cartridge at sufficient volume to expel excess coating material.

Referring now to FIG. 10 the plasma fixture 70 with said cartridge 6 inserted therein. The plasma fixture 70 is disk with an impression of said cartridge 6 with said wings 52 opened cut out of one surface of said plasma fixture 70. Said cartridge 6 is pressed into the impression in said plasma fixture 70 and is held in place by the pressure of the walls of the impression within said plasma fixture 70. An exhaust channel 72 is provided which allows the plasma to leave said cartridge 6. This process is more clear in the discussion of FIG. 11 below. In the preferred embodiment, said plasma fixture 70 is made from Repro Rubber (Trademark) distributed by Flexbar Machine Corporation of Islandia, N.Y.; but other mold making material having similar properties could be used.

Referring now to FIG. 11, a sectional view of said cartridge 6 and said plasma fixture 70 is shown. Plasma enters said plasma fixture 70 through a plasma entry 74. The plasma flows through and around the entire inside surface of said cartridge 6 and exits through the exhaust channel 72 (see FIG. 10). Because of the disclosed configuration of said plasma fixture 70; the entire inside surface of said cartridge 6 is plasma treated, but none of the outside surface is plasma treated and none of the surface of said wings 52 is treated. Because neither the top coat nor the base coat adheres effectively to areas which are not plasma treated, this method of plasma treating insures that neither coating adheres to areas of said cartridge 6 where coating is not desired.

Referring now to FIG. 12, a top view of a cartridge holder 76 with said cartridge 6 of the instant invention inserted into the cartridge holder 76 is shown. Said cartridge holder 76 is a block of polypropylene (other materials having similar characteristics may be used) with holes drilled through it to accommodate the tubular portion of said cartridge 6 and slots cut into the top to accommodate a portion of said wings 52 configured to accept said wings 52 when said wings 53 are partially open. Said wings 52 snap fit into the wing slots in said cartridge holder 76 and hold said cartridge 6 in position within said cartridge holder 76.

Referring now to FIG. 13 a sectional view of said cartridge holder 76 and said cartridge 6 of the instant invention is shown. This view, perhaps, better shows the configuration of said cartridge holder 76 and said cartridge 6 when said cartridge 6 is inserted into said cartridge holder 76. This Figure shows the apparatus used (except for the flipper which is not shown) and the position of said cartridge 6 described in steps 4 and 6 of the coating process described above.

In the preferred embodiment of the instant invention, all parts and elements of said plunger 2 and said receptor 4 are made of stainless steel, but other materials having similar characteristics may be used. It may be found that tungsten or tungsten alloys work better for certain elements. In areas where elements engage each other, such as the threaded portions of said shank 12 and said collar 24, it may be found that those areas should be permanently coated with some conventional coating to reduce friction. In the preferred embodiment, said cartridge 6 is made from polypropylene, but other materials, including metals, could be used provided they have similar properties, particularly the ability to be sterilized. In the preferred embodiment, the making material referred to in step 3 above is polyvinyl alcohol.

While preferred embodiments of this invention have been shown and described above, it will be apparent to those skilled in the art that various modifications may be made in these embodiments without departing from the spirit of the present invention.

We claim:

1. A method of applying a bilaminar coating to the interior of an object where the bilaminar coating includes a base coat which is a first material in a first solvent which will adhere to the surface of the object and a top coat which is a second material in a second solvent which will adhere or bond to the base coat, the method including the steps of:
(1) Inserting the object into a plasma fixture, the plasma fixture being configured and shaped such that areas of the surface of the object to which it is desirable that the bilaminar coating not be affixed to are masked by said plasma fixture;
(2) Applying plasma to said plasma fixture and the object such that areas of the object to which the bilaminar coating are to be adhered are treated with plasma;
(3) Removing the object from said plasma fixture and inserting the object into a holder, the holder being capable of holding the object such that the interior of the object is exposed, and said holder further being capable of being manipulated;
(4) Positioning said holder and the object such that the base coat may be introduced into the interior of the object and the base coat will flow from a first end of the object toward a second end of the object;
(5) Introducing the base coat into the object and allowing the base coat to flow from the first end of the object to the second end of the object;
(6) Flipping said holder and the object such that the base coat within the object flows from the second end of the object to the first end of the object;
(7) Inducing a flow of air through the interior of the object, the air being of sufficient volume and temperature to remove excess base coat and smooth the coating and then induce evaporation of the solvent from the base coat and removal of sufficient solvent from the base coat to cause the base coat to dry;
(8) Placing said holder and the object in an oven and applying sufficient heat to cure the base coat;
(9) Positioning said holder and the object such that the top coat may be introduced into the interior of the object and the top coat will flow from a first end of the object toward a second end of the object;
(10) Introducing the top coat into the object and allowing the top coat to flow from the first end of the object to the second end of the object;
(11) Flipping said holder and the object such that the top coat within the object flows from the second end of the object to the first end of the object;
(12) Inducing a flow of air through the interior of the object, the air being of sufficient volume and temperature to remove excess top coat and smooth the coating and then induce evaporation of the solvent from the top coat and removal of sufficient solvent from the top coat to cause the top coat to dry;
(13) Placing said holder and the object in an oven and applying sufficient heat to cure the top coat;
whereby a bilaminar coating is affixed to the interior of an object by treating the areas to which the bilaminar coating is to be applied with plasma, by applying an even coating of the base coat of the bilaminar coating to the desired surfaces of the object, by curing the base coat in a manner which prevents a skin from forming on the surface of the base coat, by applying an even coating of the top coat of the bilaminar coating to the desired surfaces of the object, and by curing the top coat in a manner which prevents a skin from forming on the surface of the top coat.

2. A method of applying a bilaminar coating to the interior of an object where the bilaminar coating includes a base coat which is a first material in a first solvent which will adhere to the surface of the object and a top coat which is a second material in a second solvent which will adhere or bond to the base coat, the method including the steps of:
(1) Inserting the object into a plasma fixture, the plasma fixture being configured and shaped such that areas of the surface of the object to which it is desirable that the bilaminar coating not be affixed to are masked by said plasma fixture;
(2) Applying plasma to said plasma fixture and the object such that areas of the object to which the bilaminar coating are to be adhered are treated with plasma;
(3) Removing the object from said plasma fixture and inserting the object into a holder, the holder being capable of holding the object such that the interior of the object is exposed, and said holder further being capable of being manipulated;
(4) Positioning said holder and the object such that the interior of the object is not horizontal;
(5) Introducing the base coat into the lower end of the object and filling the interior of the object with base coat;
(6) Draining base coat from the lower end of the object at a controlled rate such that the desired thickness of coating remains on the interior of the object;
(7) Inducing a flow of air through the interior of the object, the air being of sufficient volume and temperature to remove excess base coat and smooth the coating and then induce evaporation of the solvent from the base coat and removal of sufficient solvent to cause the base coat to dry;
(8) Placing said holder and the object in an oven and applying sufficient heat to cure the base coat;
(9) Positioning said holder and the object such that the interior of the object is not vertical;
(10) Introducing the top coat into the interior of the object from the higher end of the object and filling the interior of the object with top coat;
(11) Inducing a flow of air through the interior of the object in sufficient volume to remove excess top coat from and smooth the coating in the interior of the object;
(12) Inducing a flow of air through the interior of the object, the air being of sufficient volume and temperature to induce evaporation of the solvent from the top coat and removal of most of the solvent from the top coat;
(13) Placing said holder and the object in an oven and applying sufficient heat to cure the top coat;
whereby a bilaminar coating is affixed to the interior of an object by treating the areas to which the bilaminar coating is to be applied with plasma, by applying an even coating of the base coat of the bilaminar coating to the desired surfaces of the object, by curing the base coat in a manner which prevents a skin from forming on the surface of the base coat, by applying an even coating of the top coat of the bilaminar coating to the desired surfaces of the object, and by curing the top coat in a manner which prevents a skin from forming on the surface of the top coat.

3. The method of claim 1 in which the object is a cartridge made from a material selected from the group consisting of polypropylene, acrylic polymers, acrylic copolymers, nylon, polyester, cellulose acetate, and acetate/butyrate and the cartridge includes a loading zone at one end into which an intra-ocular lens may be loaded and a hollow tube through which the lens may be pushed into the interior of an eye.

4. The method of claim 2 in which the object is a cartridge made from a material selected from the group consisting of polypropylene, acrylic polymers, acrylic copolymers, nylon, polyester, cellulose acetate, and acetate/butyrate and the cartridge includes a loading zone at one end into which an intra-ocular lens may be loaded and a hollow tube through which the lens may be pushed into the interior of an eye.

5. The method of claim 3 in which the base coat comprises a polymer or copolymer supplied in an organic solvent and the top coat comprises a lubricious, water-soluble polymer.

6. The method of claim 4 in which the base coat comprises a polymer or copolymer supplied in an organic solvent and the top coat comprises a lubricious, water-soluble polymer.
